(19) United States
(12) Patent Application Publication
(10) Pub. No.: US 2017/0150949 A1
(43) Pub. Date: Jun. 1, 2017

(54) DEVICE AND METHOD FOR DETECTING OVULATION USING SALIVA

(71) Applicant: Medi Brain Lab Co., Ltd., Bucheon-si (KR)

(72) Inventors: Jae-Kwan LIM, Bucheon-si (KR); Ig-Mo KOO, Incheon (KR)

(73) Assignee: Medi Brain Lab Co., Ltd., Bucheon-si (KR)

(21) Appl. No.: 15/060,904

(22) Filed: Mar. 4, 2016

(30) Foreign Application Priority Data
Nov. 27, 2015 (KR) 10-2015-0167157

(51) Int. Cl. A61B 10/00; G01N 21/21; G06T 7/00; G01N 33/487

(52) U.S. Cl. CPC A61B 10/0012; G01N 33/487; G01N 21/21; G06T 7/0012; G06T 7/0081; A61B 10/0051; G01N 2201/12; G01N 2201/0683; G06T 2207/10056; G06T 2207/30004

(57) ABSTRACT

A device for detecting ovulation using saliva according to the present invention comprises: a device body having a lens disposed in the inner space thereof and a pinhole formed through a side opposite to the lens; a scattering unit coupled to one side of the device body, which has the pinhole formed therein, and configured to scatter light to be supplied to the pinhole; and a sample plate which is separably mounted in the device body so as to be located between the lens and the pinhole and on which saliva is placed, wherein the pinhole is configured to convert light, which passed through the scattering unit, into point light so as to uniformly supply the light to the sample plate, so that saliva crystals formed by drying of the saliva will form a striking contrast with the surrounding portions when the light is scattered by the saliva crystals.

### BACKGROUND OF THE INVENTION

Field of the Invention

The present invention relates to a device and method for detecting ovulation using saliva, and more particularly, to a device and method for detecting ovulation using saliva, which cart detect ovulation by supplying light uniformly to a sample plate and acquiring high-quality saliva crystallization patterns.

Description of the Prior Art

In general, medical devices are frequently used by hospitals and patients who are final consumers. In recent years, many home medical devices have been developed which used not only in hospitals, but also at home. Such home medical devices are frequently used not only at home, but also in hospitals, and are provided by hospitals or used by patients.

In recent years, the use of home medical devices not only by elders, but also by young people or middle-aged people, has increased. Such home medical devices include blood glucose meters, blood pressure meters, body thermometers, body weight scales, height meters, physical therapy machines, hearing aids, etc.

In addition, medical devices for women include ovulation predictors. Such ovulation predictors are devices required not only for women who desire pregnancy, but also for women who desire to avoid pregnancy. These ovulation predictors can simply and accurately predict ovulation cycles, and thus are used for women who desire pregnancy, and can be used without the inconvenience or additional cost incurrence caused by the use of contraceptive articles such as contraceptive drugs or condoms. Owing to such advantages, the ovulation predictors have recently been spotlighted.

Korean Utility Model Laid-Open Publication No. 20-2000-0015504 discloses an optical device of predicting women's ovulation dates using saliva, which comprises: a lipstick-shaped body case made of aluminum; a cover; a body made of plastic; a lighting unit having a push button and a replaceable battery and provided in the lower portion of each of the body and the body case; and a small-sized biological microscope separably mounted in the upper portion of the body.

In the above-described optical device, the body has a ring so that the body case and the cover are easily attached to and detached from the body, and the push button of the lighting unit protrudes outward from body case so that it conveniently pushed. The small-sized biological microscope is configured to be attached to the body, and the magnification of the microscope is controlled by a control handle so that a clear image is formed.

The above-described optical device for predicting women's ovulation dates is operated as follows. The small-sized biological microscope is separated from the body, and saliva is lightly applied to the glass of the microscope. At 2-3 minutes after application of salvia, the microscope is coupled to the body, and then the push bottom in the lower portion of the body is pushed, and the control handle is rotated from left to right so that the image becomes clear. Then, based on study results indicating that there is a definite correlation between estrogen and LH hormones, which are female hormones, follicle growth and saliva crystallization, the degree of crystallization of the saliva is measured, thereby determining the ovulation date, the transition phase and the infertile phase.

### SUMMARY OF THE INVENTION

It is an object of the present invention to provide a device for detecting ovulation using saliva, in which a pinhole is formed in a device body so that light can be uniformly supplied to a sample plate, whereby saliva crystals placed on the sample plate form a striking contrast with the surrounding portions when the light is scattered by the saliva crystals, thereby determining accurately whether the subject is ovulating. Another object of the present invention is to provide a method for detecting ovulation using saliva, which can determine whether saliva crystals correspond to an ovulation phase or how saliva crystals are near the ovulation phase, by converting a saliva crystallization pattern, which includes the saliva crystals, into a black-and-white image, calculating a determination coefficient that is the length-to-area ratio of each of portions corresponding to the saliva crystals in the black-and-white image, and applying the determination coefficient to an evaluation function.

In accordance with an embodiment of the present invention, there is provided a device for detecting ovulation using saliva, comprising: a device body having a lens provided in the inner space thereof and a pinhole formed through a side opposite to the lens; a scattering unit coupled to one side of the device body, which has the pinhole formed therein, and configured to scatter light to be supplied to the pinhole; and a sample plate which is separably mounted in the device body so as to be located between the lens and the pinhole and on which saliva is placed, wherein the pinhole is configured to convert light, which passed through the scattering unit, into point light so as to uniformly supply the light to the sample plate, so that saliva crystals formed by drying of the saliva will form a striking contrast with the surrounding portions when the light is scattered by the saliva crystals.

In this embodiment, the device for detecting ovulation using saliva may further comprise: a first polarizer disposed between the pinhole and the sample plate in the inner space; and a second polarizer located between the sample plate and the lens and disposed in the inner space so as to be parallel with the first polarizer, wherein the second polarizer is orthogonally oriented to the first polarizer.

In this embodiment, the device for detecting ovulation using saliva may further comprise: an imaging unit disposed in a straight line with the lens and configured to image the saliva crystals; and a determining unit connected to the imaging unit and configured to determine ovulation by reading saliva crystallization patterns acquired by the imaging unit.

In accordance with another embodiment of the present invention, there is provided a device for detecting ovulation using saliva, comprising: a device body having a lens disposed therein and a light inlet provided therethrough; a first polarizer disposed in the device body so as to be adjacent to the light inlet; a second polarizer disposed between the lens and the first polarizer in the device body; and a sample plate which is separably mounted between the first polarizer and the second polarizer in the device body and on which saliva is placed, wherein the second polarizer is orthogonally oriented to the first polarizer, so that light polarized by the first polarizer can't pass through the second polarizer and only light scattered by saliva crystals formed by drying of the saliva will pass through the second polarizer to the lens.

In this embodiment, the device for detecting ovulation using saliva may further comprise: an imaging unit disposed in a straight line with the lens and configured to image the saliva crystals; and a determining unit connected to the imaging unit and configured to determine ovulation by reading saliva crystallization patterns acquired by the imaging unit.

In accordance with another embodiment of the present invention, there is provided a method for detecting ovulation using saliva, comprising the steps of: (A) acquiring a saliva crystallization pattern; (B) converting the saliva crystallization pattern into a black-and-white image; (C) calculating the area and length of the black portion of the black-and-white image; (D) calculating a determination coefficient that is the ratio of the length to the area; and (E) determining whether the saliva corresponds to ovulation phase, based on the determination coefficient.

In an embodiment of the present invention, the determination coefficient may be calculated using the following equation:

R=L/A

wherein R is the determination coefficient, L is the length, and A is the area.

In an embodiment of the present invention, step (E) may comprise determining that the saliva corresponds to any one of an ovulation phase, a transition phase and an infertile phase, based on the determination coefficient.

In an embodiment of the present invention, the area in step (C) may he calculated by the total number of pixels in the black portion.

In an embodiment of the present invention, the length in step (C) may be calculated by the pixel number of the outline of the black portion, and the outline may be the boundary between the black portion and the white portion in the black-and-white image.

### BRIEF DESCRIPTION OF THE DRAWINGS

FIG. 1 schematically shows the configuration of a device for predicting ovulation using saliva according to a first embodiment of the present invention.

FIG. 2 schematically shows the configuration of a device for predicting ovulation using saliva according to a second embodiment of the present invention.

FIGS. 3 and 4 schematically show the configuration of a device for predicting ovulation using saliva according to a third embodiment of the present invention.

FIG. 5 is a schematic flowchart showing determining ovulation from saliva crystals according to a method for detecting ovulation using saliva according to an embodiment of the present invention.

FIG. 6 schematically shows three divided phases (infertile phase, transition phase and ovulation phase) of an evaluation function according to a determination coefficient.

FIG. 7 depicts saliva crystallization patterns corresponding to the infertile phase, the transition phase and the ovulation phase, respectively.

FIG. 8 depicts black-and-white images corresponding to the infertile phase, the transition phase and the ovulation phase, respectively.

### DETAILED DESCRIPTION OF THE INVENTION

Hereinafter, a device for predicting ovulation using saliva according to preferred embodiment of the present invention will be described with reference to the accompanying drawings.

Device for Detecting Ovulation Using Saliva

### First Embodiment

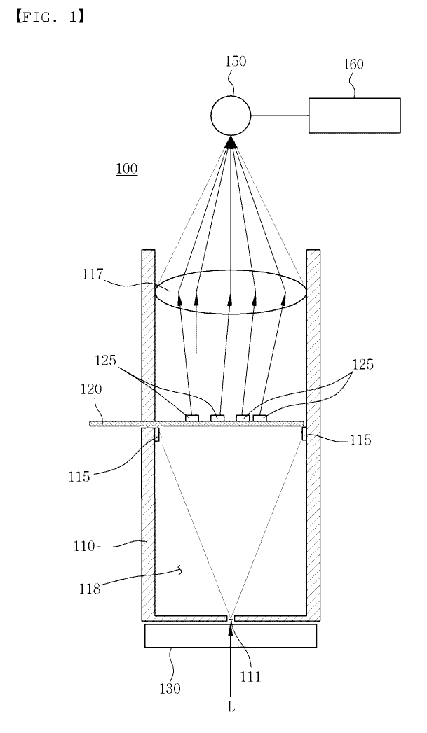

As shown in FIG. 1, a device 100 for predicting ovulation using saliva according to an embodiment of the present invention comprises a device body 110, a sample plate 120, a scattering unit 130, an imaging unit 150, and a determining unit 160.

The present invention is directed to a device for detecting ovulation using saliva crystals. As the amount of estrogen increases, the amount of saliva in female saliva increases. Thus, when saliva is applied to a sample plate and dried, the amount of the saliva crystals increases as the amount of salt in the saliva increases.

The device body 110 is configured such that light (L) is uniformly supplied to the sample plate 120 having saliva placed thereon so that saliva crystals 125 can be clearly distinguished from the surrounding portions.

The device body 110 has a cylindrical structure having an. inner space 118 provided therein. A lens 117 is mounted in the inner space 118 of the device body 110. The lens 117 serves to magnify the saliva placed on the sample plate.

The device body 110 has a pinhole 111 formed through the side opposite to the lens 117. The pinhole 111 provides a passage through which the light (L) enters the inner space 118. The pinhole 111 serves to enable the light (L), which passed through the scattering unit 130, to be converted into point light so as to be uniformly supplied to the sample plate 120.

On one side of the device body 110, the scattering unit 130 is mounted. The scattering unit 130 serves to scatter the light (L) that enters the pinhole 111. The scattering unit 130 can prevent the light (L) from being concentrated on a portion.

The device body 110 has an entrance opening through which the sample plate 120 enters the inner space 118. In addition, the inner wall surface of the device body 110 is provided with a mounting protrusion 115 configured to support the sample plate 120. The sample plate 120 is inserted into the device body 110 through the entrance opening (reference numeral not shown) and mounted on the mounting protrusion 115. The sample plate 120 has a flat plate type or film type structure that is easily inserted into and separated from the device body through the entrance opening.

On the sample plate 120, saliva is placed. The sample plate 120 is coated with a hydrophilic or heterogeneous material that serves to allow the applied saliva to be uniformly spread on the plate. Generally, when saliva is applied to a polymer film not coated with a hydrophilic or heterogeneous material, the saliva will be gathered while forming water droplets in several places on the plate by surface tension, and a crystal aggregate will be formed. However, if the sample plate is coated with a hydrophilic or heterogeneous material, saliva will be thinly and uniformly spread so that saliva crystals will be uniformly distributed on the surface of the sample plate. Examples of the hydrophilic coating material include polyurethane (PU), and examples of the heterogeneous coating material include polyethylene glycol (PEG) and silane.

The sample plate 120 is located between the lens 117 and the pinhole 111 at the focal distance of the lens 117. The sample plate 120 is separably mounted in the device body 110. The sample plate 120 is preferably made of a material that transmits the light (L) that entered the inner space 118 through the pinhole 111.

The imaging unit 150 is located in a straight line with the lens 117 and serves to image the saliva crystals 125 placed on the sample plate 120. The imaging unit 150 may also be a camera provided in a smart device such as a mobile phone. The imaging unit 150 provides the pattern data of the saliva crystals 125 to the determining unit 160.

The determining unit 160 serves to read the saliva crystallization pattern to thereby detect ovulation. The determining unit 160 has stored therein pattern data for the infertile phases, transition phases and ovulation phases of other subjects, determined using saliva. The determining unit 160 can analyze the saliva crystallization pattern of the subject in comparison with the pattern data stored therein, thereby determining whether the subject is ovulating.

According to the present invention, the light (L) scattered by the scattering unit 130 can he converted into point light through the pinhole 111, and thus can be uniformly supplied to the sample plate 120 having saliva placed thereon so that the crystals of the saliva can be clearly distinguished from the surrounding portions, whereby the imaging unit 150 can acquire high-quality saliva crystallization pattern. Therefore, according to the present invention, high-quality saliva crystallization pattern can be acquired, thus ensuring the reliability of ovulation detection.

### Second Embodiment

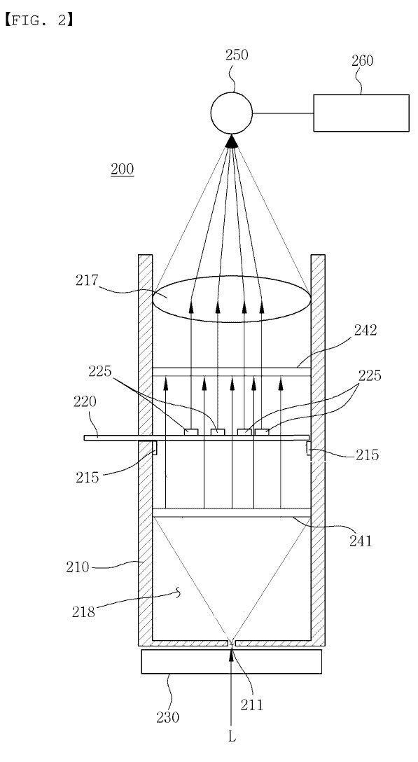

Hereinafter, a device for detecting ovulation using saliva according to a second embodiment of the present invention will be described with reference to FIG. 2.

As shown in FIG. 2, a device 200 for detecting ovulation using saliva according to a second embodiment of the present invention comprises a device body 210, a first polarizer 241, a second polarizer 242, a sample plate 220, a scattering unit 230, an imaging unit 250, and a determining unit 260.

In this embodiment, the device body 210, the sample plate 220, the scattering unit 230, the imaging unit 250 and the determining unit 260 are configured in substantially the same manner as the device body 110, sample plate 120, scattering unit 130, imaging unit 150 and determining unit 160 of the first embodiment, and thus the description thereof will be omitted to avoid repeated description.

Hereinafter, the first polarizer 241 and the second polarizer 242, which differ from the first embodiment, will be described.

The first polarizer 241 is located in an inner space 218 between a pinhole 211 and the sample plate 220. At this time, the first polarizer 241 is disposed in parallel with the sample plate 220. Herein, the sample plate 220 is located at the focal distance of a lens 217 and mounted on a mounting protrusion 215.

The second polarizer plate 242 is located between the sample plate 220 and the lens 217. The second polarizer 242 is disposed in parallel with the first polarizer plate 241 in the inner space 218.

The second polarizer 242 is orthogonally oriented to the first polarizer 241. The first polarizer 241 may be a vertical polarizer. The second polarizer 242 may be a horizontal polarizer. Light (L) polarized by the first polarizer can't pass through the second polarizer and only light (L) scattered by saliva crystals formed by drying of the saliva will pass through the second polarizer to the lens.

The light (L) enters the inner space 218 through the pinhole 211 and is supplied to the first polarizer 241. Herein, the first polarizer 241 vertically provides light (L) to the sample plate. The polarized light (L) is scattered by saliva crystals 225 in portions on which the saliva crystals 225 are placed, and the scattered light is passed through the second polarizer 242 and supplied to the lens 217. On the other hand, the polarized light (L) passed through the first polarizer 241 is supplied directly to the second polarizer 242 without being scattered in the surrounding portions the saliva crystals 225, and it is blocked by the second polarizer 242 so as to be prevented from entering the lens 217.

According to the second embodiment of the present invention, the polarized light (L) passed through the first polarizer 241 is blocked from passing through the second polarizer 242, and only the light (L) scattered by the saliva crystals 225 formed by drying of saliva is passed through the second polarizer 242 and supplied to the lens 217, whereby the imaging unit 250 can acquire high-quality saliva crystallization pattern. Therefore, according to the present invention, high-quality saliva crystallization pattern can be acquired, thus ensuring the reliability of ovulation detection.

### Third Embodiment

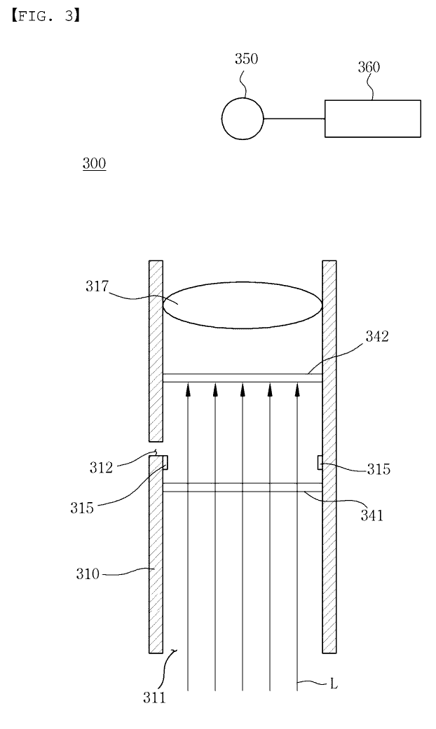

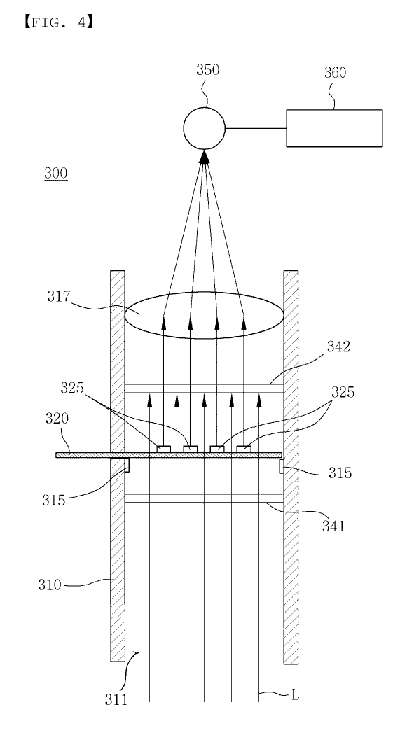

Hereinafter, a device for detecting ovulation using saliva according to a third embodiment of the present invention will be described with reference to FIGS. 3 and 4.

As shown in FIGS. 3 and 4, a device 300 for detecting ovulation using saliva according to a third embodiment of the present invention comprises a device body 310, a first polarizer 341, a second polarizer 342, a sample plate 320, an imaging unit 350, and a determining unit 360. Herein, the sample plate 320, the imaging unit 350 and the determining unit 360 perform substantially the same functions as those of the sample plate 120, the imaging unit. 150 and the determining unit 160 of the first embodiment as described above, and thus the description thereof will be omitted in this embodiment in order to avoid repeated description.

The device body 310 has a cylindrical structure having a light inlet 311 provided at one side thereof. A lens 317 is M disposed in the device body 310. The lens 317 is disposed in the device body 310 so as to be opposite to the light inlet 311. The lens 317 serves to magnify saliva placed on the sample plate 320.

The device body 310 has an entrance opening 312 through which the sample plate 320 enters the inner space. On the inner wall surface of the device body 310, a mounting protrusion 311 for supporting the sample plate 320 is provided. The sample plate 320 is inserted into the device body 310 through the entrance opening 312 and is mounted on the mounting protrusion 315. The sample plate 320 has a flat plate type or film type structure that is easily inserted into and separated from the device body 310 through the entrance opening 312.

The sample plate 320 is located at the focal distance of the lens 317. Saliva is placed on the sample plate 320. The sample plate 320 is preferably made of a material that transmits the light (L) that entered the internal space through a pinhole 311.

The first polarizer 341 is located at the inner space between the light inlet 311 and the sample plate 320. Herein, the first polarizer 341 is disposed in parallel with the sample plate 320. The first polarizer 341 may be a vertical polarizer. The light (L) passes vertically through the first polarizer 341.

The second polarizer 342 is located between the sample plate 320 and the lens 317. The second polarizer 342 is disposed in parallel with the first polarizer 341 in the inner space. The second polarizer 342 may be a horizontal polarizer. The light polarized by the first polarizer 341 can't pass through the second polarizer 342 because the second polarizer 342 is orthogonally oriented to the first polarizer 341.

In this embodiment, the first polarizer 341 and the second polarizer 342 are orthogonally oriented. The light (L) supplied to the device body 310 through the light inlet 311 moves along a light path through the first polarizer 341 and the second polarizer 342 as shown in FIGS. 3 and 4. FIG. 3 illustrates the path of the light (L) in the case in which the sample plate 320 is not mounted in the device body 310, and FIG. 4 illustrates the path of the light (L) in the case in which the sample plate 320 is mounted in the body 310.

As shown in FIG. 3, among the light (L) supplied to the device body 310 through the light inlet 311, only a light (L) that passes vertically through the first polarizer 341 is supplied to the second polarizer 342. The second polarizer 342 has a structure that passes horizontally through polarized light (L), and thus the polarized light (L) passed through the first polarizer 341 is blocked by the second polarizer 342 so as to be prevented from entering the lens 317.

On the other hand, in the case in which the sample plate 320 having saliva placed thereon is mounted in the device body 310, the light (L) introduced into the device body 310 through the light inlet 311 moves along a light path as shown in FIG. 4.

As shown in FIG. 4, the light (L) introduced into the device body 310 through the light inlet 311 is supplied to the first polarizer 341. Herein, the first polarizer 341 vertically transmits a light(L) to the sample plate 320. The polarized light (L) is scattered by the saliva crystals 325 in portions having the saliva crystals 325 placed thereon, and are passed through the second polarizer 342 to the lens 317.

On the other hand, the polarized light (L) passed through the first polarizer 341 enters directly the second polarizer 342 without being scattered in the surrounding portions of the saliva crystals 325, and are blocked from entering the lens 317 by the second polarizer 342.

According to the third embodiment of the present invention, the polarized light (L) passed through the first polarizer 341 is blocked from entering the second polarizer 342, and only the light (L) scattered by the saliva crystals 325 formed by drying of saliva is passed through the second polarizer 342 to the lens 317, whereby the imaging unit 350 can acquire high-quality saliva crystallization pattern. Therefore, according to the present invention, high-quality pattern rata of the saliva crystals can be acquired, thus ensuring the reliability of ovulation detection.

Method for Detecting Ovulation Using Saliva

A method for detecting ovulation using saliva according to an embodiment of the present invention comprises the steps of: acquiring a saliva crystallization pattern; converting the saliva crystallization pattern into a black-and-white image; calculating the area and length of the black portion of the black-and-white image; calculating a determination coefficient that is the ratio of the length to the area; and determining whether the saliva corresponds to ovulation, based on the determination coefficient.

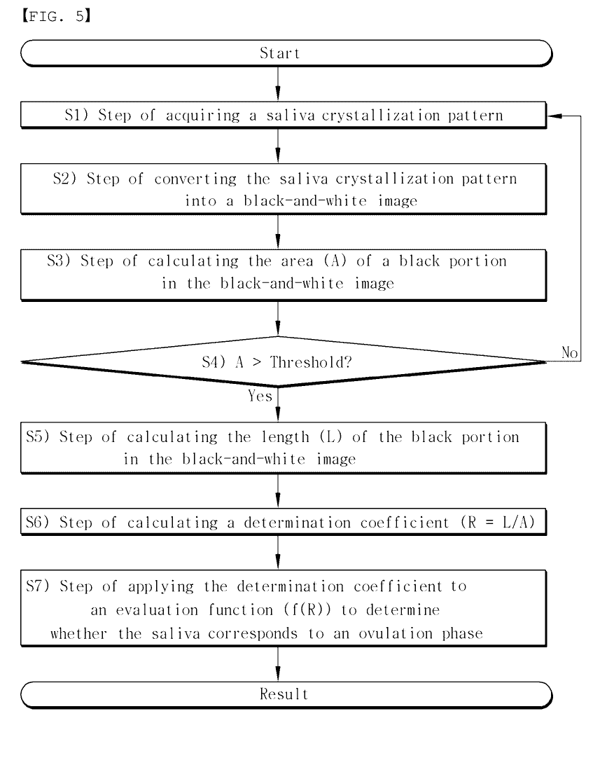

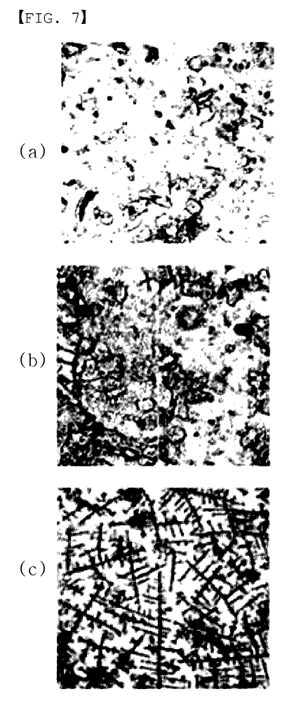

More specifically, referring to FIG. 5, a saliva crystallization pattern is acquired by the device 100, 200 or 300 for detecting ovulation using saliva according to the present invention (A). Namely, saliva placed on the sample plate 120 is imaged by the imaging unit 150, thereby acquiring a saliva crystallization pattern. The saliva crystallization pattern is divided into saliva crystals and portions surrounding the saliva crystals. FIG. 7(a) shows a saliva crystallization pattern of the infertile phase, FIG. 7(b) shows a saliva crystallization pattern of the transition phase, and FIG. 7(c) shows a saliva crystallization pattern of the ovulation phase. As shown in FIG. 7(a), the saliva in the saliva crystallization pattern of the infertile phase is non-crystalline and has an irregular shape. On the contrary, as can be seen in FIG. 7(c), the saliva in the saliva crystallization pattern of the ovulation phase is crystalline and has a characteristic elongate shape. Namely, it can be seen that the shape of saliva crystals in the ovulation phase has a small area and a long length compared to the shape of saliva crystals in the infertile or transition phase.

In step S2, the saliva crystallization pattern acquired by the imaging unit 150 is provided to the determining unit 160. In the determining unit 160, the saliva crystallization pattern is converted into a black-and-white image. The reason why the saliva crystallization pattern is converted into the black-and-white image is to classify the saliva crystals and the surrounding portions as black and white to thereby facilitate calculation of the area and length of the saliva crystals.

Step S2 is subdivided into preprocessing, binarization image processing, and noise processing with a morphological erosion and dilation algorithm.

Herein, preprocessing is to clarify saliva crystals and the surrounding portions by color correction such as brightness correction and/or contrast correction in the original of the saliva crystallization pattern acquired by the imaging unit 150.

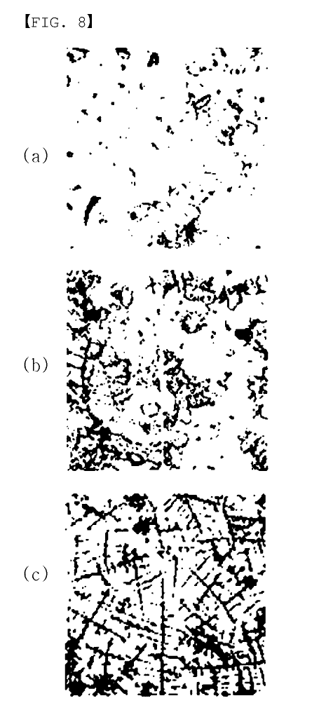

Binarization image processing is to convert the preprocessed saliva crystallization pattern into a black-and-white image. In the black-and-white image, portions corresponding to the saliva crystals are indicated as black, and portions corresponding to the portions surrounding the saliva crystals are indicated as white. FIG. 8(a) shows a black-and-white image of the infertile phase, FIG. 8 (b) shows a black-and-white image of the transition phase, and FIG. 8(c) shows a black-and-white image of the ovulation phase.

Next, the black-and-white image obtained by binarization image processing is denoised by a morphological erosion and dilation algorithm preset in the determining unit 160. Herein, the morphological erosion and dilation algorithm is known in the art, and thus the detailed description thereof is omitted in this embodiment.

By this denoising, the shape of the black portions corresponding to the saliva crystals in the black-and-white image are clearly distinguished from the shape of the white portions corresponding to the portions surrounding the saliva crystals, so as to enable the determining unit 160 to accurately calculate the area (A) of the black portions in step S3 and the length (L) of the outlines in step S5, thereby more accurately determining whether the saliva crystals correspond to the ovulation phase or whether the saliva crystals correspond to the time near the ovulation phase.

Next, in step S3, the area (A) of the black portions in the black-and-white image is calculated by the determining unit 160. When the black-and-white image is processed in pixel units, the area (A) corresponds to the total number of pixels in each of the outlines of the black portions. However, it is to be understood that this description is illustrative only and the unit indicating the area (A) can be changed in various ways depending an image processing method within a scope obvious to those skilled in the art. As used herein, the term “outline” refers to the boundary between the black portion and the white portion in the black-and-white image.

In step S4, the determining unit 160 determines that, if the total area (A) of the black portions in the black-and-white image is smaller than the threshold value, the position of the sample plate 120 having saliva placed thereon is not proper or saliva is not placed within the imaging range of the imaging unit 150 due to a problem such as background lighting. Thus, the position of the sample plate 120 is adjusted so that the saliva crystals will be easily visible to the imaging unit 150, after which step S1 to step S3 may be performed again.

Herein, the threshold value is not associated with the evaluation of the infertile phase, the transition phase, the ovulation phase, etc., as described below, and is influenced by the pixel number of the image acquired by the imaging unit 150. In other words, the threshold value is a fixed constant value that determines whether information contained in the saliva crystallization pattern is over a level adequate for determination of the ovulation phase. For example, the threshold value may correspond to a value at which the black portions account for 5% or more of the pixel number of the camera image.

On the other hand, if the total area (A) of the black portions is greater than the threshold value, it is determined that the saliva crystallization pattern was properly acquired by the imaging unit 150, and then the length (L) of the black portion corresponding to each of the saliva crystals in the black-and-white image is calculated (step S5).

Herein, the length (L) of the black portion in the black-and-white image corresponds to the pixel number of the outline of each of the black portions. As described above, the term “outline” refers to the boundary between the black portion and the white portion in the black-and-white image.

Next, in the determining unit 160, a determination coefficient. (R=L/A) is calculated (step S6). Herein, the area (A) is the total area of the black portion corresponding to each of the saliva crystals in step S3, and the length (L) refers to the total length of the outline of the black portion, which corresponds to the boundary between the black portion and the white portion in the black-and-white image.

As the number of the saliva crystals in the black-and-white image increases, the determination coefficient (R) value of the black-and-white image increases. Meanwhile, the black-and-white image is a digital image which is expressed as a group of square pixel units, and thus the determination coefficient (R) can be calculated using the following equation 1:

R=L/A Equation 1

wherein R is the determination coefficient, L is the length of the black portion, and A is the area of the black portion.

In other words, the determination coefficient (R) is the ratio of the total pixel number of the outline of each of the black portions to the total pixel number of the area of each of the black portions. The determination coefficient (R) approaches 1 as the saliva crystals have a more elongate shape, and approaches 0 as the saliva crystals have a more irregular shape. The determination coefficient (R) is calculated in the range of 0<R<1. The determination coefficient (R) has a greater value, that is, a value approaching 1, as the saliva is nearer the ovulation period.

Finally, in the determining unit 160, the determination coefficient (R) is applied to an evaluation function (f(R)) in step S7, thereby determining whether the saliva crystals correspond to the ovulation phase. The evaluation function (f(R)) is a function preset in the determining unit 160.

The evaluation function (f(R)) is used to determine whether the saliva crystals correspond to the infertile phase (0<R<T1), the transition phase (T1<R<T2) or the ovulation phase (T2<R<1) according to the determination coefficient (R). Herein, T1 and T2, which correspond to criteria for determination, are values preset by a statistical method, and are in the range of 0<T1<T2<1.

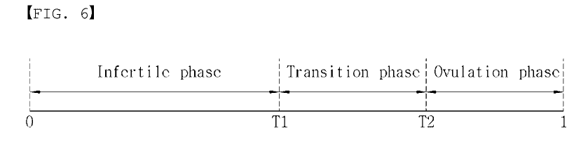

For example, as shown in FIG. 6, if the determination coefficient (R) calculated in step 36 is smaller than T1 preset in the determining unit 160, the determining unit 160 determines that the saliva crystals correspond to the infertile phase. In addition, if the determination coefficient (R) calculated in step 36 is greater than T2 and near 1, the determining unit 160 determines that the saliva crystals correspond to the ovulation phase. Furthermore, the determining unit 160 can determine how the saliva is near the ovulation period, based on information calculated by the evaluation function.

As described above, according to the present invention, ovulation can be detected by providing light uniformly to the sample plate and acquiring high-quality saliva crystallization pattern.

In addition, the pinhole is provided in the device body so that light can be uniformly supplied to the sample plate, whereby the saliva crystals placed on the sample plate form a striking contrast with the surrounding portions when the light is scattered by the saliva crystals, thereby determining accurately whether the subject is ovulating.

Additionally, according to the invention, a saliva crystallization pattern is converted into a black-and-white image by a binarization image processing technique so that saliva crystals and the portions surrounding the saliva crystals are simplified into black and white. Also, the length (L) and area (A) of each of the black portions, which are the variables of the determination coefficient, are simply calculated by calculating the pixel number of the outline of each of the black portions and the number of pixels inside the outline of each of the black portions. Thus, it is possible within a short time to determine whether the saliva crystallization pattern corresponds to the ovulation phase or how the saliva crystallization pattern is near the ovulation period.

1. A device for detecting ovulation using saliva, comprising:
a device body having a lens disposed in an inner space thereof and a pinhole formed through a side opposite to the lens;
a scattering unit coupled to one side of the device body, which has the pinhole formed therein, and configured to scatter light to be supplied to the pinhole; and
a sample plate which is separably mounted in the device body so as to be located between the lens and the pinhole and on which saliva is placed,
wherein the pinhole is configured to convert light, which passed through the scattering unit, into point light so as to uniformly supply the light to the sample plate, so that saliva crystals formed by drying of the saliva will form a striking contrast with portions surrounding the saliva crystals when the light is scattered by the saliva crystals.

2. The device of claim 1, further comprising:
a first polarizer disposed between the pinhole and the sample plate in the inner space; and
a second polarizer located between the sample plate and the lens and disposed in the inner space so as to be parallel with the first polarizer,
wherein the second polarizer is orthogonally oriented to the first polarizer.

3. The device of claim 1, further comprising:
an imaging unit disposed in a straight line with the lens and configured to image the saliva crystals; and
a determining unit connected to the imaging unit and configured to determine ovulation by reading saliva crystallization pattern acquired by the imaging unit.

4. A device for detecting ovulation using saliva, comprising:
a device body having a lens disposed therein and a light inlet provided therethrough;
a first polarizer disposed in the device body so as to be adjacent to the light inlet;
a second polarizer disposed between the lens and the first polarizer in the device body; and
a sample plate which is separably mounted between the first polarizer and the second polarizer in the device body and on which saliva is placed,
wherein the second polarizer is orthogonally oriented to the first polarizer, so that light polarized by the first polarizer can't pass through the second polarizer and only light scattered by saliva crystals formed by drying of the saliva will pass through the second polarizer to the lens.

5. The device of claim 4, further comprising:
an imaging unit disposed in a straight line with the lens and configured to image the saliva crystals; and a determining unit connected to the imaging unit and configured to determine ovulation by reading saliva crystallization pattern acquired by the imaging unit.

6. The device of claim 1, wherein a surface of the sample is coated with a hydrophilic or heterogeneous material.

7. A method for detecting ovulation using saliva, comprising the steps of:
(A) acquiring a saliva crystallization pattern;
(B) converting the saliva crystallization pattern into a black-and-white image;
(C) calculating an area and length of a black portion of the black-and-white image;
(D) calculating a determination coefficient that is a ratio of the length to the area; and
(E) determining whether the saliva corresponds to ovulation phase, based on the determination coefficient.

8. The method of claim 7, wherein the determination coefficient may be calculated using the following equation:
R=L/A
wherein R is the determination coefficient, L is the length, and A is the area.

9. The method of claim 7, wherein step (E) comprises determining that the saliva corresponds to any one of an ovulation phase, a transition phase and an infertile phase, based on the determination coefficient.

10. The method of claim 7, wherein the area in step (C) is calculated by a total number of pixels in the black portion.

11. The method of claim 7, wherein the length in step (C) is calculated by a pixel number of an outline of the black portion, and the outline is a boundary between the black portion and a white portion in the black-and-white image.
